(19) United States
(12) Patent Application Publication
(10) Pub. No.: US 2023/0181302 A1
(43) Pub. Date: Jun. 15, 2023

(54) IMPLANT

(71) Applicant: OLYMPUS WINTER & IBE GMBH, Hamburg (DE)

(72) Inventors: Thorsten JÜRGENS, Hamburg (DE); Thomas WOSNITZA, Lüneburg (DE); Henning SCHWEINBERGER, Basthorst (DE)

(73) Assignee: OLYMPUS WINTER & IBE GMBH, Hamburg (DE)

(21) Appl. No.: 18/076,775

(22) Filed: Dec. 7, 2022

Related U.S. Application Data
(60) Provisional application No. 63/289,842, filed on Dec. 15, 2021.

(51) Int. Cl. A61F 2/04; A61M 29/00

(52) U.S. Cl. CPC A61F 2/04; A61M 29/00; A61F 2002/047; A61F 2210/0014; A61F 2250/0059

(57) ABSTRACT

An implant in which the Lobus medius (median lobe) can be treated in a simple and reliable manner. This is achieved by virtue of the fact that an implant for the treatment of a urinary tract has a wire structure. The wire structure has a loop wire, which can be expanded to form a loop, wherein at least two net wires can be stretched in this loop and the wire structure can be placed around the median lobe.

The invention relates to an implant in accordance with the preamble of claim 1.

Various methods and techniques are known for the treatment of the urinary tract, in particular benign prostatic enlargement (benign prostatic hyperplasia, BPH). In a minimally invasive, particularly gentle treatment of BPH symptoms, a removable implant is temporarily placed in the urethra or in the prostatic portion of the urethra of the patient. Such an implant is a wire structure made of a shape memory alloy, such as, for example. Nitinol. In a folded state, the wire structure is pushed into the correct position by a catheter in order to unfold there into its predetermined basic structure. This structure, which can be formed from three or four wires, is a basket structure. This basket structure widens the urethra. Owing to the expansion of the wire structure against the tissue of the urethra, the tissue of the urethra is denatured over the course of a few days. This denaturation of the tissue takes place on account of the ischemic pressure of the individual wires on the cells of the tissue, leading to reduced or completely absent blood flow. As a result, the lack of blood supply leads to a lack of oxygen in the cells and ultimately to death of the cells. Within a few days, the tissue can be reduced to such an extent that the urinary flow almost normalizes. After completion of this treatment, the implant can be recovered from the urethra by means of a catheter.

By means of the technique described above, the tissue in the urethra can be treated in a very efficient manner. Owing to the enlargement of the prostate, the Lobus medius (median lobe) may also protrude a long way into the interior of the bladder. it is conceivable that the bladder outlet will be displaced as a result of this protrusion of the median lobe, but also as a result of a possible enlargement of the median lobe. However, this displacement of the bladder outlet by protrusion and/or by enlargement of the median lobe can also occur independently of an enlargement of the prostate. For medical reasons, it is important to avoid displacement of the bladder outlet. However, known methods and techniques for this are rather complex and traumatizing for the patient. The technique described above is also not suitable for treating the urinary tract in an appropriate way.

Proceeding therefrom, the object of the invention is that of providing an implant with which the Lobus medius (median lobe) can be treated in a simple and reliable manner.

An implant for achieving this object has the features of claim 1. Accordingly, it is envisaged that an implant for the treatment of a urinary tract has a wire structure. Treatment is carried out by applying a local ischemic pressure to the tissue of a urinary organ, in particular of the median lobe and, if appropriate, at the same time of the urethra. In a folded state, this implant can be introduced with a distal end in the lead into the urethra, where it unfolds for the treatment of the tissue. There, pressure is exerted on the tissue by the individual wires of the wire structure. The invention envisages that the wire structure has a loop wire which can be expanded to form a loop, wherein at least two net wires can be stretched in this loop and the wire structure can be laid around the median lobe. In the unfolded state, the implant assumes the shape of a tennis racket, wherein the loop wire forms the frame of the racket and the net wires form the stringing. For the treatment of the median lobe, this loop wire is guided around the median lobe, wherein at least one end of the net wires is held loosely. In the process, the shape of the tennis racket changes to a shape resembling a trap net. As soon as the implant or the loop formed by the loop wire is guided over the median lobe, both the loop wire and the net wires are tightened, with the result that the loop circumference is reduced and the net wires are tensioned. As a result of this tightening, both the loop wire and the net wires exert the ischemic pressure on the tissue, as a result of which the tissue denatures within a few days. Following treatment, the loop can be opened again and removed from the median lobe. This minimally invasive procedure allows the median lobe to be treated in a particularly simple, efficient and gentle manner.

Provision is preferably made for a multiplicity of net wires to be stretched within the loop, wherein the net wires are oriented parallel and/or transversely, in particular perpendicularly, to one another. By using a plurality of net wires, for example 2, 3, 4, 5 or 6, it is possible to produce a plurality of pressure points on the median lobe, leading to even more efficient use of the implant. By means of this enlargement of the pressure surface, a larger amount of tissue can be denatured in the same period of time. By changing the orientation of the net wires within the loop, the stability of the loop can be increased. Moreover, an oblique arrangement of the net wires enables the tissue to be subjected to a pressure over its entire surface.

A further preferred exemplary embodiment of the invention can provide for one end of the net wires and of the loop wire to be firmly fastened to the wire structure and another end of the net wires and of the loop wire to be guided loosely and in a tensionable manner to the proximal end of the implant and to be connected there, in particular releasably, to a handling device. The ends can be fastened by clamping, adhesive bonding, crimping, knotting or the like, for example. For handling, the loose ends can be brought together to form a wire, for example, or are led out to the outside individually through the urethra. It is also conceivable for the loose ends to be movable by means of the handling device. By pulling on the loose ends of the wires, these can be tensioned. By releasing the wires, the loop structure or the network structure can be opened, thereby enabling the implant to be easily removed from the median lobe.

Provision can be made for the handling device to be a shaft, a wire, a thread or the like. Depending on the application, the implant can first of all be introduced into the body and positioned there by means of this handling device. It is then conceivable for the handling device to be able to be decoupled from the implant. For recovery and/or for readjusting or retensioning the wires, the handling device can be recoupled to the proximal end of the implant. It is likewise conceivable for the handling device to be permanently connected to the implant.

A particularly advantageous exemplary embodiment of the invention can provide for one or more connecting springs to be drawn onto the loop wire, at least in some section or sections, and for the net wires to be threaded through these connecting springs in order to fix the position. Thus, one embodiment envisages that the loop wire forming the loop is guided completely through a guide spring and the net wires are guided, preferably symmetrically, through this spring at an opposite position on the loop in order in this way to form the tennis racket-like shape. The connecting springs are designed in such a way that they can just accommodate the loop wire. An alternative exemplary embodiment envisages that the loop wire has short connecting springs only in some section or sections. These short sections are fixed in their position on the loop wire to ensure that the net wires, which are guided through the connecting springs, remain in their predetermined position and do not slip into a tangle with one another. As the loop wire is spread out to form the loop, the connecting spring, because of its property as a spring, follows the enlargement of the loop circumference. Likewise, the connecting spring contracts when the loop wire is tightened, thus enabling it also to be pulled back into the catheter. During treatment, the individual wire sections of the connecting spring have a reinforcing effect on the denaturation of the tissue.

It is furthermore possible to envisage, according to the invention, that the loop wire be designed as a connecting spring. In this exemplary embodiment, there is no loop wire. To tighten the loop, the connecting springs are correspondingly tightened. For the technique described above, the connecting spring too can be produced from a shape memory material.

A preferred exemplary embodiment of the present invention is explained in greater detail below with reference to the drawing. In this drawing:

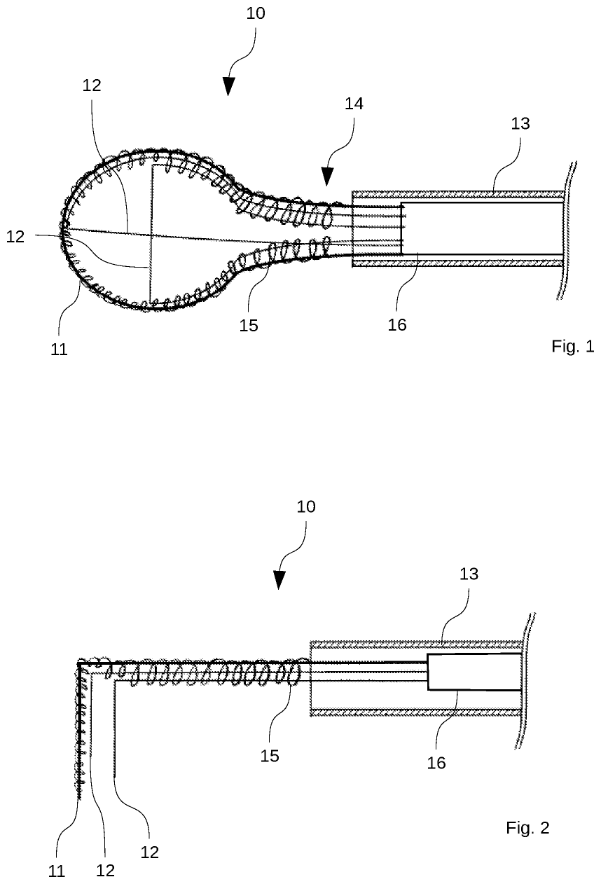

FIG. 1 shows a schematic illustration of an exemplary embodiment of an implant, and

FIG. 2 shows a schematic side view of the exemplary embodiment according to FIG. 1.

One possible exemplary embodiment of the invention is illustrated schematically in the figures. It should be expressly pointed out that there is no intention to restrict the invention to this exemplary embodiment. Rather, it is envisaged that the invention can also be implemented by other embodiments.

According to the exemplary embodiment of the implant 10 according to the invention illustrated schematically in FIGS. 1 and 2, it is formed by an encircling loop wire 11, which forms a loop. At least two net wires 12 are stretched in this loop or loop wire 11. These net wires 12 are arranged within the loop like the strings of a tennis racket, Ideally, the two net wires 12 are oriented transversely or perpendicularly to one another within the bent loop wire 11.

In the exemplary embodiment illustrated in FIG. 1, a net wire 12 crosses the loop formed by the loop wire 11, two further net wires 12 being positioned perpendicularly thereto. It is also conceivable for further net wires 12 to be arranged parallel to these net wires 12 within the loop wire 11.

The implant 10 is inserted into the urinary tract through a catheter 13, which is illustrated only schematically. During this process, the loop wire 11 and the net wires 12 are initially folded together within the catheter 13. By being pushed out of the catheter 13, the loop wire 11 unfolds to form the loop, and the net wires 12 are stretched within the loop. This unfolding is achieved through the choice of the material of the loop wire 11. This is because the loop wire 11 is preferably produced from a shape memory material, for example Nitinol. Both the loop wire 11 and net wires 12 can be fastened or releasably fastened by at least one of their ends to a handling device 16. This handling device 16 may be a rod, a shaft, a wire or a thread. By means of this handling device 16, the implant 10 can be introduced into the body and pulled out again. It is conceivable here for the handling device 16 to be capable of being releasably coupled to the implant 10. Thus, during treatment, the handling device 16 can be detached from the implant 14. Particularly for recovery of the implant 10, said implant can be recoupled to the handling device 16.

In each case one end of the loop wire 11 and of the net wires 12 is fixed to a proximal end 14 of the implant 10, in particular to the handling device 16. The second ends are guided back loosely to the proximal end 14 of the implant 10. There, they can be moved back and forth by pulling and can preferably be fixed or fixed to the handling device 16. By exerting the pull, it is possible, for example, to reduce the circumference of the loop wire 11. The mechanical tension of the net wires 12 can furthermore be maintained by simultaneously pulling on the free ends of the net wires 12. For adequate mechanical stability, the net wires 12 are produced, in particular, from titanium or a spring steel.

In order to fix the net wires 12 within the loop of the loop wire 11, the net wires 12 are guided along an inner side of the loop wire 11 and are held in position there by fastening means. This ensures that the net wires 12 retain their position relative to one another as the loop diameter is reduced and increased. An advantageous exemplary embodiment of the invention envisages that the loop wire 11 is surrounded by a connecting spring 15. This connecting spring 15 can either extend over the entire length of the loop wire 11 or only over a section or sections.

In the exemplary embodiment illustrated in FIGS. 1 and 2, the connecting spring 15 extends over the entire length of the loop wire 11. The individual turns of the connecting spring 15 enable the net wires 12 to be threaded through. This is a simple and reliable way of ensuring that the net wires 12 retain their position relative to one another. Owing to the multiplicity of turns in the connecting springs 15, it is also possible to stretch a multiplicity of net wires 12 in almost any orientations and combinations in the loop. However, it has been found that, for reasons of space, two to four net wires 12 within the loop achieve a particularly advantageous effect on the tissue.

For the treatment of the median lobe, the loop formed by the loop wire 11 is guided over the median lobe. During this process, the loose end of the net wires 12 is initially not fixed. As a result, the net wires 12 and also the loop wire 11 come to lie around the median lobe. In the next step, both the loop wire 11 and the net wires 12 are then tightened. This changes both the circumference of the loop and the mechanical load exerted by the net wires 12 on the surrounded tissue. During the next few days, the already described denaturation of the tissue of the median lobe takes place. After completion of the treatment, the loose end of the loop wire 11 and of the net wires 12 is released and lifted off from the median lobe. When the implant 10 or the net wires 12 are pulled back, the loose ends of said wires hang out of the plane of the loop, as illustrated in FIG. 2. In this state, the implant 10 can be removed from the urinary tract in a simple and gentle manner.

### LIST OF REFERENCE SIGNS

10 implant

11 loop wire

12 net wire

13 catheter

14 proximal end

15 connecting spring

16 handling device

1. An implant, for the treatment of a urinary tract of a person by applying a local ischemic pressure to the tissue of a median lobe by means of a wire structure, wherein the implant can be introduced into the urethra in a folded state with a distal end in the lead and unfolds in the urethra to form the wire structure in order to treat the tissue, wherein the wire structure has a loop, wire, which can be expanded to form a loop, wherein at least two net wires can be stretched in this loop and the wire structure can be placed around the median lobe.

2. The implant as claimed in claim 1, wherein a multiplicity of net wires can be stretched within the loop, wherein the net wires are oriented parallel and/or transversely, to one another.

3. The implant as claimed in claim 1, wherein one end of the net wires and of the loop is firmly fastened to the wire structure and another end is guided loosely and in a tensionable manner to the proximal end of the implant and is connected there, in particular releasably, to a handling device.

4. The implant as claimed in claim 3, wherein the handling device can be a shaft, a wire, a thread or the like.

5. The implant as claimed in claim 1, wherein the length of the loop and of the net wires can be varied in order in this way to exert a pressure on the tissue and to release the tissue again.

6. The implant as claimed in claim 1, wherein one or more connecting springs are drawn onto the loop wire, at least in some section or sections, and the net wires are threaded through the connecting springs in order to fix the position.

7. The implant as claimed in claim 1, wherein the loop is designed as a connecting spring.

8. The implant as claimed in claim 1, wherein all the wires are brought together at a proximal end of the implant, and the loose wires can be fixed there or can be led out to the outside through the urethra.
